(19) United States
(12) Patent Application Publication
(10) Pub. No.: US 2018/0223439 A1
(43) Pub. Date: Aug. 9, 2018

(54) PARTICLE-PARTICLE INTERACTION USING ACOUSTIC WAVES

(71) Applicant: FloDesign Sonics, Inc., Wilbraham, MA (US)

(72) Inventors: BART LIPKENS, BLOOMFIELD, CT (US); WALTER M. PRESZ, JR., WILBRAHAM, MA (US); JASON DIONNE, SIMSBURY, CT (US); RUI TOSTOES, WILBRAHAM, MA (US); GOUTAM GHOSHAL, SOUTH GRAFTON, MA (US)

(21) Appl. No.: 15/947,746

(22) Filed: Apr. 6, 2018

Related U.S. Application Data
(63) Continuation-in-part of application No. 15/232,194, filed on Aug. 9, 2016.
Application No. 15/232,194 is a continuation of application No. 14/221,527, filed on Mar. 21, 2014, now Pat. No. 9,410,256.
Application No. 14/221,527 is a division of application No. 12/947,757, filed on Nov. 16, 2010, now Pat. No. 8,691,145.
(60) Provisional application No. 62/641,234, filed on Mar. 9, 2018.
(60) Provisional application No. 62/482,681, filed on Apr. 6, 2017.
(60) Provisional application No. 61/261,686, filed on Nov. 16, 2009.
(60) Provisional application No. 61/261,676, filed on Nov. 16, 2009.

(51) Int. Cl. C25B 9/00; A61L 2/025; C01B 13/11; C02F 1/36; C12M 1/00; C25B 1/13

(52) U.S. Cl. CPC C25B 9/00; C02F 2303/04; C02F 2209/008; A61L 2/025; C01B 13/11; C02F 1/4672; C12M 47/02; C25B 1/13; C02F 1/36

(57) ABSTRACT

Methods for causing interaction between two sets of particles are disclosed herein. The two sets of particles are co-located in a multi-dimensional acoustic standing wave, or are co-located by acoustic streaming. This is more effective than conventional methods, for example by increasing the homogeneity of the fluid mixture containing the particles, while using reduced amounts of one particle set.

### CROSS-REFERENCE TO RELATED APPLICATIONS

This application claims priority to U.S. Provisional Patent Application Ser. No. 62/641,234, filed on Mar. 9, 2018, and to U.S. Provisional Patent Application Ser. No. 62/482,681, filed on Apr. 6, 2017. This application is also a continuation-in-part of U.S. patent application Ser. No. 15/232,194, filed on Aug. 9, 2016, which is a continuation of U.S. patent application Ser. No. 14/221,527, filed Mar. 21, 2014, now U.S. Pat. No. 9,410,256, which was a divisional of U.S. patent application Ser. No. 12/947,757, filed Nov. 16, 2010, now U.S. Pat. No. 8,691,145, which claimed priority to U.S. Provisional Patent Application No. 61/261,686, filed on Nov. 16, 2009, and U.S. Provisional Patent Application No. 61/261,676, filed on Nov. 16, 2009, all of which are incorporated, herein, by reference in their entireties.

### BACKGROUND

The present disclosure relates to methods that cause at least two sets of particles to interact with each other, using acoustic waves. Such methods may be useful in cell therapy applications such as cell-antibody conjugation, cell-bead incubation, and viral transduction/transfection.

Biotechnology and bioprocessing of materials have many applications in a number of fields, including medicine, food and beverage and agriculture, to name a few. Condensing particles or fluids is a useful process in a number of fields. Functionalized beads or microcarriers can be employed in a number of useful techniques for cell culturing, cell separation, or other bioprocesses or in other applications in other fields. Other useful operations include mixing biomaterials to achieve certain results, or manipulating biomaterials spatially, such as positioning them within a three-dimensional space.

### BRIEF DESCRIPTION

The present disclosure relates, in various embodiments, to acoustic devices that may be used in a number of applications in a number of fields for biomaterials. The acoustic devices may operate on particles or droplets of fluid, referred to herein collectively as particles, or may operate on fluid mixtures. Particles may include cells or biomaterial produced by cells, such as proteins, monoclonal antibodies or vesicles, for example. Particles may also or otherwise include beads or microcarriers. An acoustic device for dispersing particles throughout a host fluid is described. An acoustic device for moving particles to specified locations or for positioning particles in three dimensions is described.

Disclosed herein in various embodiments are methods for causing particle-particle interactions between first particles and second particles. The first particles and the second particles are placed in an acoustophoretic device, for example by placing the particles in a bag that is inserted into the acoustophoretic device, or by flowing a fluid mixture containing the particles through the acoustophoretic device. The acoustophoretic device comprises: an acoustic chamber in which the first particles and the second particles are placed; and an ultrasonic transducer and a reflector opposite the ultrasonic transducer, the ultrasonic transducer including a piezoelectric material that can be driven to create a multi-dimensional acoustic standing wave in the acoustic chamber. The ultrasonic transducer is driven to create the multi-dimensional acoustic standing wave. As a result, the first particles and the second particles are co-located by the multi-dimensional acoustic standing wave. Put another way, the first particles and the second particles are placed in close enough proximity to each other to permit reactions between each other.

The first particles and the second particles may be suspended in a fluid. Such fluids can include cell culture media, water, saline solution, and the like.

In particular embodiments, the first particles are cells, and the second particles are selected from the group consisting of antibodies, beads, and viruses. In particular embodiments, the cells are Chinese hamster ovary (CHO) cells, NSO hybridoma cells, baby hamster kidney (BHK) cells, human cells, regulatory T-cells, helper T-cells, cytotoxic T-cells, memory T-cells, effector T-cells, gamma delta T-cells, Jurkat T-cells, CAR-T cells, B cells, or NK cells, peripheral blood mononuclear cells (PBMCs), algae, plant cells, bacteria, or viruses. The reactions between these particles can include cell-antibody conjugation, cell-bead incubation, and viral transduction or transfection.

The ultrasonic transducer may be driven for a time period of about 5 minutes to about 15 minutes. The ultrasonic transducer may be driven at a frequency of about 3 MHz to about 20 MHz. In some embodiments, the frequency of the multi-dimensional acoustic standing wave is varied in a sweep pattern to move the first particles relative to the second particles.

The piezoelectric material of the ultrasonic transducer may be lead zirconate titanate (PZT) or lithium niobate. The acoustophoretic device may further comprise a cooling unit for cooling the ultrasonic transducer.

Also disclosed are methods for causing first particles to interact with second particles, comprising: placing the first particles and the second particles in an acoustophoretic device comprising: an acoustic chamber in which the first particles and the second particles are placed; and an ultrasonic transducer and a reflector opposite the ultrasonic transducer, the ultrasonic transducer including a piezoelectric material; and driving the ultrasonic transducer to cause acoustic streaming; wherein the acoustic streaming causes interaction between the first particles and the second particles.

These and other non-limiting characteristics are more particularly described below.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations.

FIG. 1 is a diagram illustrating a method/process of the present disclosure, in which the efficiency of viral transduction is enhanced. A cell culture is combined with a viral vector for green fluorescent protein (GFP) and exposed to acoustic processing, where the multi-dimensional acoustic standing wave brings the cells and viruses into close proximity with each other, enhancing reaction efficiency. After washing and overnight incubation, GFP is expressed.

FIG. 2A is an exploded perspective view of an example acoustophoretic device according to the present disclosure including a cooling unit for cooling the transducer. FIG. 2B is a perspective view of the assembled device of FIG. 2A.

FIG. 3 is a perspective view of another acoustophoretic device that can be used to practice the methods/processes of the present disclosure. A disposable container, such as a plastic bag, contains fluid mixture with two particle types that are caused to interact with each other in a separate acoustophoretic device containing one or more ultrasonic transducers.

FIG. 4 is a cross-sectional diagram of a conventional ultrasonic transducer.

FIG. 5 is a cross-sectional diagram of an ultrasonic transducer of the present disclosure. An air gap is present within the transducer, and no backing layer or wear plate are present.

FIG. 6 is a cross-sectional diagram of an ultrasonic transducer of the present disclosure. An air gap is present within the transducer, and a backing layer and wear plate are present.

FIG. 7 is a graph of electrical impedance amplitude versus frequency for a square transducer driven at different frequencies.

FIG. 8 illustrates the trapping line configurations for seven of the resonance frequencies (minima of electrical impedance amplitudes) of FIG. 7 from the direction orthogonal to fluid flow.

FIG. 9 is a computer simulation of the acoustic pressure amplitude (right-hand scale in Pa) and transducer out of plane displacement (left-hand scale in meters). The text at the top of the left-hand scale reads “×10⁻⁷”. The text at the top of the left-hand scale by the upward-pointing triangle reads “1.473×10⁻⁶”. The text at the bottom of the left-hand scale by the downward-pointing triangle reads “1.4612×10⁻¹⁰”. The text at the top of the right-hand scale reads “×10⁶”. The text at the top of the right-hand scale by the upward-pointing triangle reads “1.1129×10⁶”. The text at the bottom of the right-hand scale by the downward-pointing triangle reads “7.357”. The triangles show the maximum and minimum values depicted in this figure for the given scale. The horizontal axis is the location within the chamber along the X-axis, in inches, and the vertical axis is the location within the chamber along the Y-axis, in inches.

FIG. 10 shows the In-Plane and Out-of-Plane displacement of a crystal where composite waves are present.

FIG. 11 shows a graph illustrating a frequency sweep used to translate trapped particles along the direction of an acoustic field.

FIG. 12 is a picture of a plastic bag in which T-cells and viruses are interacting with each other.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named component and allowing the presence of other components. The term “comprising” should be construed to include the term “consisting of”, which allows the presence of only the named component, along with any impurities that might result from the manufacture of the named component.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

It should be noted that many of the terms used herein are relative terms. For example, the terms “upper” and “lower” are relative to each other in location, i.e. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, i.e. ground level. However, these terms should not be construed to require structures to be absolutely parallel or absolutely perpendicular to each other. For example, a first vertical structure and a second vertical structure are not necessarily parallel to each other. The terms “top” and “bottom” or “base” are used to refer to surfaces where the top is always higher than the bottom/base relative to an absolute reference, i.e. the surface of the earth. The terms “upwards” and “downwards” are also relative to an absolute reference; upwards is always against the gravity of the earth.

The present application refers to “the same order of magnitude.” Two numbers are of the same order of magnitude if the quotient of the larger number divided by the smaller number is a value of at least 1 and less than 10.

The acoustophoretic devices discussed herein may operate in a multimode or planar mode. Multimode refers to generation of acoustic waves by an acoustic transducer that create acoustic forces in three dimensions. The multimode acoustic waves, which may be ultrasonic, can be generated by a single acoustic transducer, and are sometimes referred to herein as multi-dimensional or three-dimensional acoustic standing waves. Planar mode refers to generation of acoustic waves by an acoustic transducer that create acoustic forces substantially in one dimension, e.g. along the direction of propagation. Such acoustic waves, which may be ultrasonic, that are generated in planar mode are sometimes referred to herein as one-dimensional acoustic standing waves.

The acoustic transducers may comprise a piezoelectric material, such as lead zirconate titanate (PZT) or lithium niobate. Such acoustic transducers can be electrically excited to generate planar or multimode acoustic waves. The three-dimensional acoustic forces generated by multimode acoustic waves include radial or lateral forces that are unaligned with a direction of acoustic wave propagation. The lateral forces may act in two dimensions. The lateral forces are in addition to the axial forces in multimode acoustic waves, which are substantially aligned with the direction of acoustic wave propagation. The lateral forces can be of the same order of magnitude as the axial forces for such multimode acoustic waves. The acoustic transducer excited in multimode operation may exhibit a standing wave on its surface, thereby generating a multimode acoustic wave. The standing wave on the surface of the transducer may be related to the mode of operation of the multimode acoustic wave. When an acoustic transducer is electrically excited to generate planar acoustic waves, the surface of the transducer may exhibit a piston-like action, thereby generating a one-dimensional acoustic standing wave. Compared to planar acoustic waves, multimode acoustic waves exhibit significantly greater particle trapping activity on a continuous basis with the same input power. One or more acoustic transducers may be used to generate combinations of planar and multi-dimensional acoustic standing waves.

Acoustophoresis is a low-power, no-pressure-drop, no-clog, solid-state approach to particle separation from fluid dispersions. The scattering of the acoustic field off the particles results in a three-dimensional acoustic radiation force, which acts as a three-dimensional trapping field. The acoustic radiation force is proportional to the particle volume (e.g., the cube of the radius) when the particle is small relative to the wavelength. The acoustic radiation force is proportional to frequency and the acoustic contrast factor. The acoustic radiation force scales with acoustic energy (e.g., the square of the acoustic pressure amplitude). For harmonic excitation, the sinusoidal spatial variation of the force is what drives the particles to the stable positions within the standing waves. When the acoustic radiation force exerted on the particles is stronger than the combined effect of fluid drag force and buoyancy/gravitational force, the particle is trapped within the acoustic standing wave field. The action of the lateral and axial acoustic forces on the trapped particles results in formation of tightly packed clusters through concentration, clustering, clumping, agglomeration and/or coalescence of particles that, when reaching a critical size, settle continuously through enhanced gravity for particles heavier than the host fluid or rise out through enhanced buoyancy for particles lighter than the host fluid. Additionally, secondary inter-particle forces, such as Bjerkness forces, aid in particle agglomeration.

The acoustic standing waves create localized regions of high and low pressure. Particles are pushed to the standing wave nodes or antinodes depending on their compressibility and density relative to the surrounding fluid. Particles of higher density and compressibility move to the nodes in the standing waves, while secondary phases of lower density move to the antinodes. The force exerted on the particles also depends on their size, with larger particles experiencing larger forces. The magnitude of the force depends on the particle density and compressibility relative to the fluid medium, and increases with the particle volume.

For purposes of the present disclosure, biological cells can be considered as particles. Most biological cell types present a higher density and lower compressibility than the medium in which they are suspended, so that the acoustic contrast factor between the cells and the medium has a positive value. As a result, the axial acoustic radiation force (ARF) drives the cells towards the standing wave pressure nodes. The axial component of the acoustic radiation force drives the cells, with a positive contrast factor, to the pressure nodes, whereas cells or other particles with a negative contrast factor are driven to the pressure anti-nodes. The radial or lateral component of the acoustic radiation force is the force that traps the cells. The radial or lateral component of the ARF is larger than the combined effect of fluid drag force and gravitational force.

Additional theoretical and numerical models have been developed for the calculation of the acoustic radiation force for a particle without any restriction as to particle size relative to wavelength. These models also include the effect of fluid and particle viscosity, and therefore are a more accurate calculation of the acoustic radiation force. The models that were implemented are based on the theoretical work of Yurii llinskii and Evgenia Zabolotskaya as described in AIP Conference Proceedings, Vol. 1474-1, pp. 255-258 (2012). Additional in-house models have been developed to calculate acoustic trapping forces for cylindrical shaped objects, such as the “hockey pucks” of trapped particles in the standing wave, which closely resemble a cylinder.

Desirably, the ultrasonic transducer(s) generates a multi-dimensional standing wave in the fluid that exerts a lateral force on the suspended particles to accompany the axial force. Typical results published in literature state that the lateral force is two orders of magnitude smaller than the axial force. In contrast, the technology disclosed in this application provides for a lateral force to be of the same order of magnitude as the axial force. However, in certain embodiments described further herein, the device use both transducers that produce multi-dimensional acoustic standing waves and transducers that produce planar acoustic standing waves. The lateral force component of the total acoustic radiation force (ARF) generated by the ultrasonic transducer(s) of the present disclosure is significant and is sufficient to overcome the fluid drag force at linear velocities of up to 1 cm/s, and to create tightly packed clusters, and is of the same order of magnitude as the axial force component of the total acoustic radiation force.

The present disclosure relates to methods of using such acoustophoretic devices containing ultrasonic transducers to bring one or more sets of particles, such as at least two sets of different particles, together. These two sets of different particles are referred to herein as “first particles” and “second particles”. Examples of such particles can include cells, antibodies, beads, and viruses. If desired, more than two different particle sets or types can also be present to be interacted with each other.

In this regard, many industrially and commercially relevant biological processes employ the mixing of various materials to cause reactions between the materials. For example, transduction is the process by which a foreign nucleic acid is introduced into a cell by a viral vector (e.g. a virus, natural or modified). The viral vector and the cell are placed in close proximity. For example, they are co-located, so that the viral vector can transfer to the cell. Current transduction processes can have relatively high cost, low efficiency, and poor ability to be scaled up for commercialization. The methods described herein can reduce cost, increase efficiency, and have a scalable platform for commercialization.

In the methods of the present disclosure, the first particles and second particles are placed in the acoustic chamber of an acoustophoretic device. Generally, they are suspended in a fluid to form a fluid mixture. The acoustophoretic device contains an acoustic chamber that has an ultrasonic transducer and a reflector opposite the ultrasonic transducer (e.g. on opposite walls of the chamber). The ultrasonic transducer includes a piezoelectric material that can be driven to create a multi-dimensional acoustic standing wave within the acoustic chamber.

The acoustophoretic force created by the acoustic standing wave on the particles can be sufficient to overcome the fluid drag force exerted by the moving fluid on these particles. In other words, the acoustophoretic force can act as a mechanism that traps the first particles and second particles in the acoustic field. The acoustophoretic force can drive the first and second particles to the stable locations of minimum acoustophoretic force amplitudes. These locations of minimum acoustophoretic force amplitudes can be the nodes of a standing acoustic wave. Over time, the collection of particles at the nodes grows steadily. Within some period of time, which can be minutes or less depending on the concentration of the particles, the collection of particles can assume the shape of a beam-like collection of disks formed from the particles. Each disk can be spaced by a half wavelength of the acoustic field.

In some embodiments, the acoustic standing wave traps the first particles and the second particles and co-locates them, improving the efficiency of reactions between the first and second particles. A number of different mechanisms may be implemented for these embodiments. In one such mechanism, the first particles and the second particles may have similar acoustic contrast factors, such that both types of particles are driven to the nodes or anti-nodes of the standing wave. This brings the two types of particles in close spatial proximity with each other more efficiently than reliance on Brownian motion (as with conventional stirring). Put another way, the two types of particles are trapped in a small three-dimensional volume created by the multi-dimensional acoustic standing wave, relative to the size of the acoustic chamber. In particular embodiments, the first particles and the second particles either both have a positive acoustic contrast factor, or both have a negative acoustic contrast factor. Put another way, their acoustic contrast factors have the same sign.

In the other mechanism, one of the two types of particles may be driven to the nodes, while the other type of particles is driven to the anti-nodes. However, at higher frequencies, the nodes and anti-nodes are sufficiently close to each other that the two types of particles can react with each other. In such embodiments, the first particles or the second particles have a positive acoustic contrast factor, and the other set of particles has a negative acoustic contrast factor. Put another way, their acoustic contrast factors have opposite signs. The particles with a positive contrast factor are driven to the nodes, and the particles with a negative contrast factor are driven to the anti-nodes. The relevant factors for this reaction mechanism include the sizes of the first particles and the second particles, and the frequency at which the ultrasonic transducer is operated.

Eventually, as the first and second particles continue to be captured and concentrated, they can attain a size and weight such that gravitational settling will occur, wherein the clusters of particles will fall out of the acoustic standing wave to the bottom of the acoustic chamber. New collections of particles can then be trapped and reacted within the acoustic field generated by the acoustic standing waves.

In addition, or alternatively, in some examples, the ultrasonic transducer is driven to cause acoustic streaming within the acoustic chamber. Briefly, acoustic streaming refers to the fluid flow that results within the acoustic chamber when the fluid absorbs the acoustic energy that is transmitted by the ultrasonic transducer (from the vibration of the ultrasonic transducer). The velocity of the fluid is induced by the oscillating acoustic waves generated by the ultrasonic transducer. Typically, when acoustic streaming is generated, it results in circulatory motion or vortices that can cause stirring in the fluid mixture. This phenomenon is nonlinear, and can cause the first particles and the second particles to interact with each other.

The first particles and the second particles are brought into proximity so that they can react with each other. In the present disclosure, the terms “interact” and “react” are used to indicate that a physical change occurs in the first particles or the second particles. For example, when the particles are cells and viruses, the virus may penetrate into the cell. As another example, when the particles are cells and beads, the bead may become bonded to the surface of the cell.

As mentioned above, examples of the first and second particles can include cells, antibodies, beads, and viruses. Examples of cells include Chinese hamster ovary (CHO) cells, NSO hybridoma cells, baby hamster kidney (BHK) cells, human cells, regulatory T-cells, helper T-cells, cytotoxic T-cells, memory T-cells, effector T-cells, gamma delta T-cells, Jurkat T-cells, CAR-T cells, B cells, or NK cells, peripheral blood mononuclear cells (PBMCs), algae, plant cells, bacteria, or viruses. The cells may be attached to microcarriers. Examples of beads include polymer beads, magnetic beads, superparamagnetic beads, and microspheres. These can be used for biochemical reactions or for labeling purposes. For example, suspension array beads include a plurality of polymeric beads wherein each type of microsphere bead has a unique identification based on variations in optical properties, typically fluorescence. The differently labeled microsphere beads further include a receptor molecule such as a DNA oligonucleotide probe, an antibody, protein or peptide. The receptor molecule, for example, binds an antigen of interest. Suspension array panels can be used to detect biomarkers for a range of maladies and bodily processes such as cancer and organ function. Probe-target hybridization is detected by detecting optically labeled targets which can determine the relative abundance of each target in the sample using flow cytometry, for example. Antibodies and viruses can be used.

Without being limited by theory, it is believed that the frequency of the multi-dimensional acoustic standing wave determines the diameter of the particles that can be trapped by the acoustic standing wave. For example, for a 2 MHz wave, the particle size is about 1 to about 100 microns.

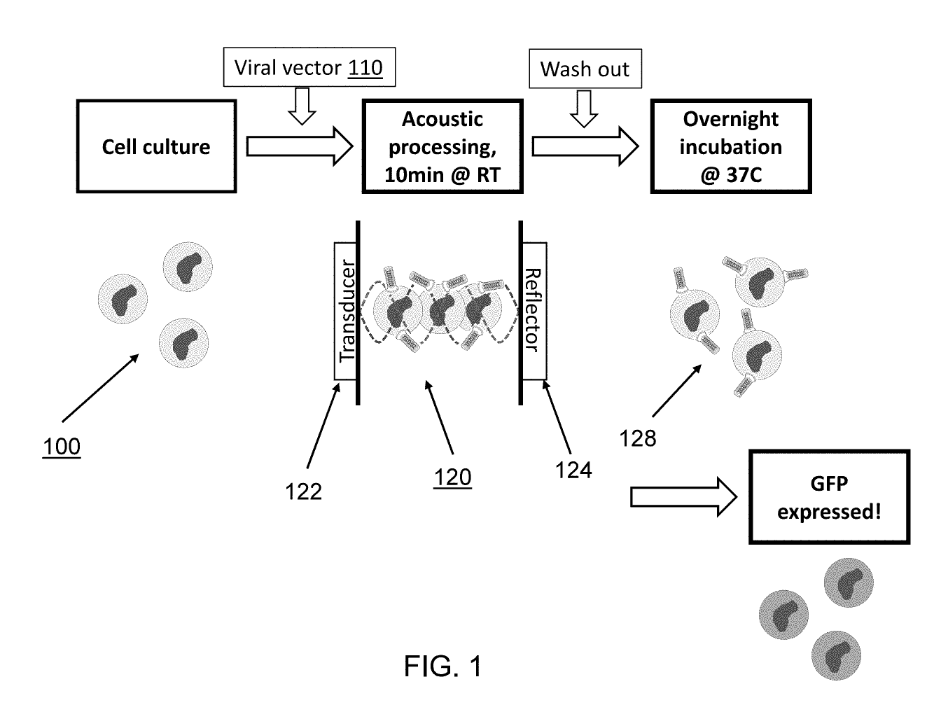

FIG. 1 is a diagram illustrating the methods of the present disclosure, as applied to viral transduction. In this example, cells are labeled with green fluorescent protein (GFP). Starting from the left-hand side of the figure, first, a cell culture 100 is combined with a viral vector 110. The fluid mixture containing the cells and the viruses are then placed in an acoustic chamber 120, which is located between an ultrasonic transducer 122 and a reflector 124. Acoustic standing waves are generated for 10 minutes at room temperature. As illustrated here, the cells and the viruses are trapped in the acoustic standing waves. The cells are trapped at the nodes and the viruses are trapped at the anti-nodes. However, due to their relative size, the cells and viruses are co-located, and the viruses are able to infect the cells (identified with reference numeral 128). After washing to remove unreacted material, the cells are incubated overnight at 37° C. and GFP is expressed in labeled cells. A similar method can be used to make T-cells that express chimeric antigen receptors (CARs), or CAR T-cells.

The methods of the present disclosure can be carried out in a continuous process, wherein a fluid mixture containing the first particles and the second particles suspended in a host fluid is flowed through the acoustophoretic device.

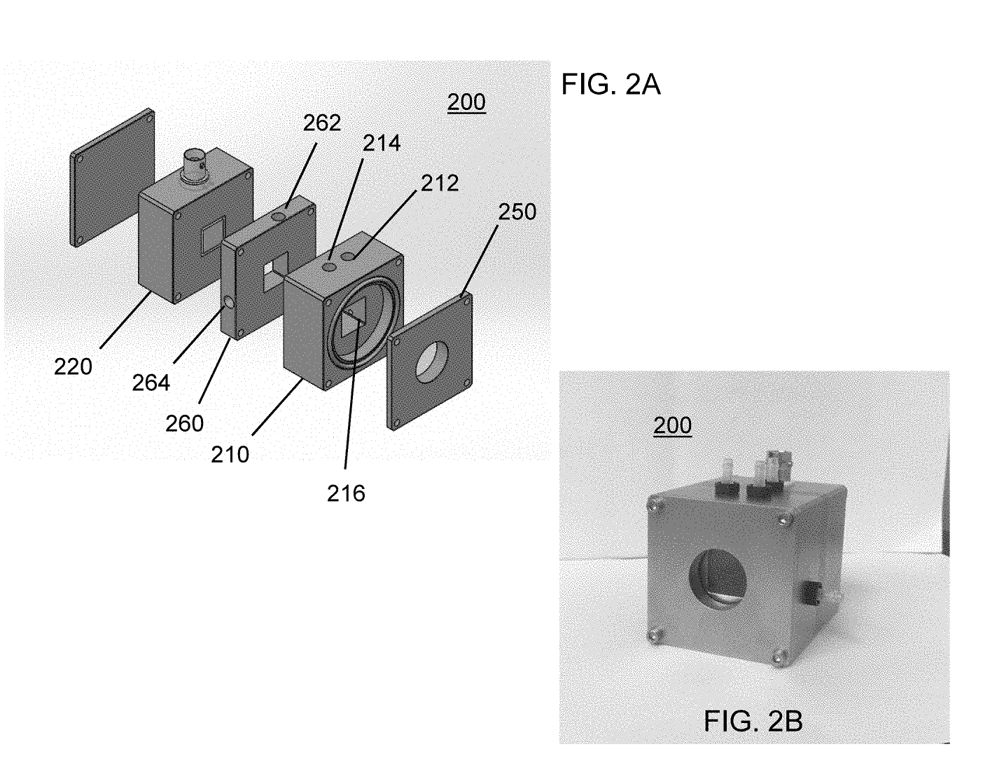

FIG. 2A is an exploded view of an acoustophoretic device 200 that can be used for continuous processing. FIG. 2B is a view of the device 200 in a fully assembled condition.

Referring to FIG. 2A, the acoustophoretic device can be built such that each component is modular, and can be changed or switched out separately from each other. Thus, when new revisions or modifications are made to a given component, the component can be replaced while the remainder of the device stays the same.

The device includes an ultrasonic transducer 220 and a reflector 250 on opposite walls of an acoustic chamber 210. It is noted that the reflector 250 may be made of a transparent material, such that the interior of the flow chamber 210 can be seen. The ultrasonic transducer is proximate a first wall of the acoustic chamber. The reflector is proximate a second wall of the acoustic chamber or can make up the second wall of the acoustic chamber.

A cooling unit 260 can be located between the ultrasonic transducer 220 and the flow chamber 210. As illustrated here, the cooling unit 260 includes an independent flow path that is separate from the flow path through the acoustic chamber. A coolant inlet 262 permits the ingress of a cooling fluid into the cooling unit. The coolant and waste heat exit the cooling unit through a coolant outlet 264. The coolant that flows through the cooling unit can be any appropriate fluid. For example, the coolant can be water, air, alcohol, ethanol, ammonia, or some combination thereof. The coolant can be a liquid, gas, or gel. The coolant can be an electrically non-conductive fluid to prevent electric short-circuits.

Alternatively, the cooling unit can be in the form of a thermoelectric generator, which converts heat flux (i.e. temperature differences) into electrical energy using the Seebeck effect, thus removing heat from the flow chamber. Put another way, electricity can be generated from undesired waste heat while operating the acoustophoretic device.

The cooling unit can be used to cool the ultrasonic transducer, which can be particularly advantageous when the device is to be run continuously with repeated processing and recirculation for an extended period of time (e.g., perfusion). Alternatively, the cooling unit can also be used to cool the fluid running through the acoustic chamber 210. For desired applications, the cell culture should be maintained around room temperature (˜20° C.), and at most around 28° C. This is because when cells experience higher temperatures, their metabolic rates increase. Without a cooling unit, however, the temperature of the cell culture flowing through the acoustic chamber can rise as high as 34° C.

It is noted that the acoustic chamber 210 is illustrated here as including at least an inlet 212 and an outlet 214. This provides access to the interior volume 216 of the acoustic chamber. Additional inlets and outlets (e.g. fluid inlet, concentrate outlet, permeate outlet, recirculation outlet, bleed/harvest outlet) may be included as desired. The interior volume 216 can be considered as being bounded by the ultrasonic transducer 220, the cooling unit 260, the acoustic chamber 210, and the reflector 250.

The flow direction of the acoustophoretic device 200 can be oriented in a direction other than horizontal. For example, the fluid flow can be vertical either upward or downward or at some angle relative to vertical or horizontal. More than one transducer can be included in the system.

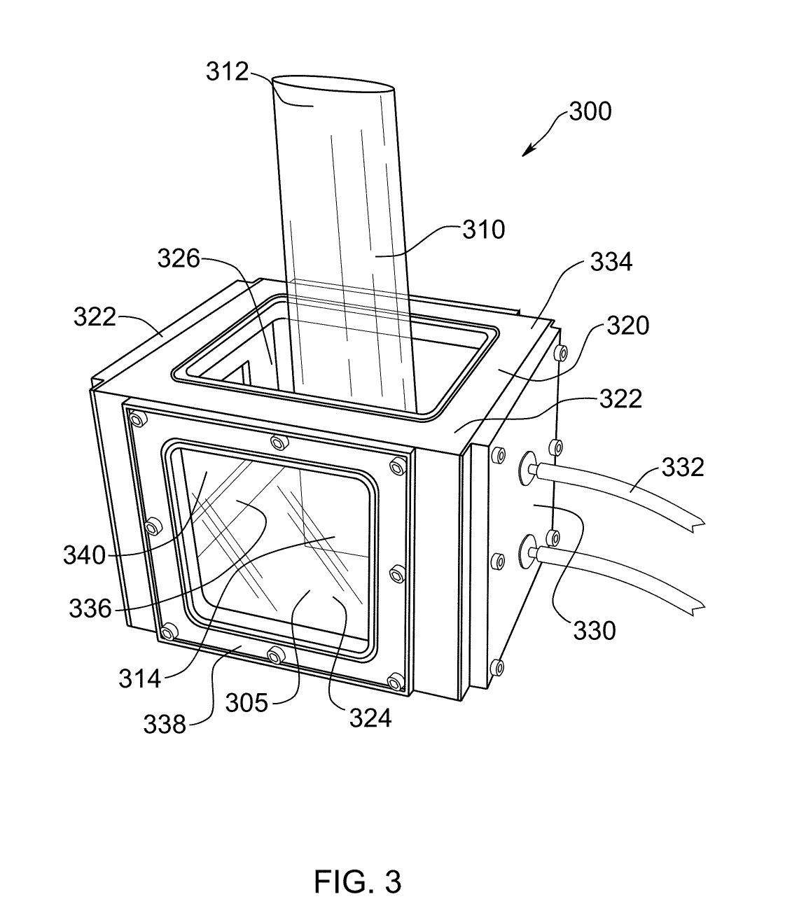

FIG. 3 illustrates another acoustophoretic device 300 which can be used to practice the methods and processes of the present disclosure. Very generally, the system includes the acoustophoretic device 300 and a substantially acoustically transparent container 310. These two components are separable from each other.

The container 310 of the acoustophoretic device is generally formed from a substantially acoustically transparent material such as plastic, glass, polycarbonate, low-density polyethylene, and high-density polyethylene (all at an appropriate thickness). However, the container may be formed from any material suitable for allowing the passage of the acoustic standing wave(s) of the present disclosure therethrough. The container may be in the form of a bottle or a bag. The difference between these forms lies in their composition and structure. A bottle is more rigid than a bag. When empty, a bag is generally unable to support itself, while a bottle is able to stand upright. For example, the container 310 shown here is a high-density polyethylene bag. Container 310 generally has an upper end 312 and a lower end 314, and an interior volume in which the fluid mixture (containing the first particles and second particles in a host fluid) is located.

The acoustophoretic device 300 is defined by at least one wall 332, and usually a plurality of walls, which form its sides. For example, the acoustophoretic device may be in the shape of a cylinder, or in a rectangle (as depicted). The wall(s) are solid. An opening 326 is present in an upper end of the acoustophoretic device, for receiving the container 310 therethrough. Again, the acoustophoretic device 300 is separable from the container 310, so that the container can be either disposable or reusable, depending upon the desired application of the acoustophoretic device. As illustrated here, the base of the acoustophoretic device 300 is solid.

The acoustophoretic device 300 includes at least one ultrasonic transducer 330 on a wall 334. The ultrasonic transducer 330 has a piezoelectric material driven by a voltage signal to create an acoustic standing wave. Cables 332 are illustrated for transmitting power and control information to the ultrasonic transducer 330. A reflector 340 may be present, and is located on the wall 336 opposite the ultrasonic transducer 330. The standing wave is thus generated through initial waves radiated from the transducer and reflected waves from the reflector. In some embodiments, a reflector is not necessary and, rather, ambient air may be used to reflect the incident waves and create the standing waves. It is to be understood that various transducer and reflector combinations may be used. The planar and/or multi-dimensional acoustic standing wave(s) are generated within the container, and are used to cause interaction of the particles within the container 310. It should be noted that there is no contact between the ultrasonic transducer and the fluid mixture within the container 310.

In certain embodiments, the acoustophoretic device includes a plurality of ultrasonic transducers 330 located on a common wall 334 opposite the wall 336 on which the reflector 340 is located. Alternatively, the ultrasonic transducers can be located opposite each other, with no reflector being present. Additionally, the acoustophoretic device 300 may include a viewing window 324 in another wall 338. As illustrated here, when a viewing window is provided, it can be in a wall adjacent the walls upon which the ultrasonic transducer(s) and reflector are located, such that the lower end 314 of the container 310 can be viewed through the viewing window 324 in the separation chamber 320. In other embodiments, the viewing window can take the place of the reflector.

In certain embodiments, a fluid, such as water, may be placed in the interstitial space 305 between the container 310 and the acoustophoretic device 300, such that the acoustic standing wave passes through both the fluid in the interstitial space and the fluid mixture in the container. The interstitial fluid can be any fluid, though it should have an acoustic impedance value that allows for good transmission of the acoustic standing wave(s), and preferably a low acoustic attenuation.

In particular embodiments, the ultrasonic transducer is driven at a frequency of about 3 MHz to about 20 MHz (megahertz). Higher frequency standing wave fields result in steeper pressure gradients, which in turn are better suited for trapping smaller particles like viruses. The ultrasonic transducer can be driven for a time period of about 5 minutes to about 15 minutes. This is a considerably shorter time period than, for example, conventional viral transduction processes where the cell culture and viral vector are incubated together for about 30 minutes to about 120 minutes. Such lengthy incubation periods are due to the reaction between cells and viruses only occurring when Brownian motion brings them in proximity to each other. Using the acoustophoretic devices of the present disclosure greatly increases the probability of cells and viruses being in sufficient proximity to react with each other. This results in higher reaction efficiency using fewer particles.

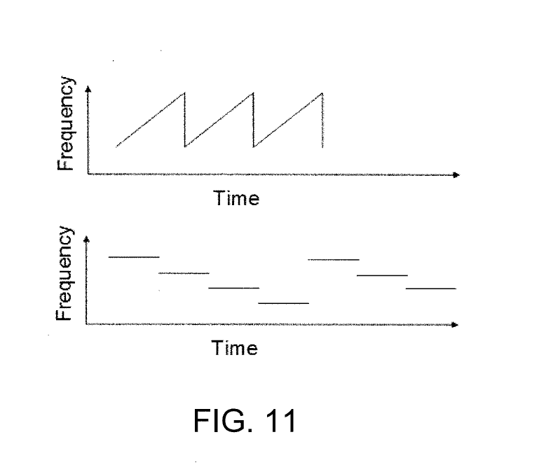

In additional embodiments, the frequency of the multi-dimensional acoustic standing wave can be varied in a sweep pattern to move the first particles relative to the second particles. This can also be used to bring the particles in sufficient proximity to react with each other. The frequency of the acoustic standing wave can be slowly swept over a small frequency range spanning at least a range of two times the frequency corresponding to the lowest-order standing wave mode of the acoustic chamber. The sweep period can be, in one example, on the order of one second. This frequency sweeping method can slowly translate the trapped particles in the direction of the acoustic field towards one of the walls of the acoustic chamber. This sweep is illustrated in FIG. 11.

FIG. 11 shows graphs of a frequency sweep or modulation used to translate trapped particles along the direction of an acoustic field. In the top graph, a saw toothed line is shown representing the variation of the frequency of the drive signal applied to the transducer over time. The increasing frequency over time with each interval that starts with a lower frequency and increases to a higher frequency represents a relatively slow frequency sweep. The relatively slow frequency sweeping method may be used to translate the particles or cells in the acoustic standing wave in the direction of propagation of the wave. For example, the frequency of the acoustic standing wave is slowly swept over a small frequency range, which spans at least a range of two frequencies corresponding to the one lower than and one higher than the resonance of the standing wave mode of the cavity or acoustic chamber. The sweep period can be on the order of seconds, however, a sweep period of less than a second or greater than tens of seconds may be used. This frequency sweeping method will slowly translate the collected microorganisms in the direction of the acoustic field towards one of the walls of the flow chamber where the particles or cells are concentrated. The concentrated cells may be may be collected for further processing, for example by being swept into a pocket in the wall of the acoustic chamber, or by removing the acoustic standing wave to permit the concentrated cells to drop into a collection chamber. It will be appreciated that an array or differing types of transducers can be used (which in turn may operate at different or varying resonance frequencies). The sweeping technique operates by shifting the nodes and/or antinodes of the acoustic standing wave in the direction of the acoustic wave by changing the frequency of the acoustic wave. As the frequency shifts through resonance modes, the particles or cells in the nodes or antinodes translate in the direction of the acoustic standing wave, for example, toward or away from the transducer. Frequency shifts toward lower frequencies can translate particles or cells towards the transducer, and frequency shifts toward higher frequencies can translate particles or cells away from the transducer.

The bottom graph in FIG. 11 shows frequency steps that change over time to periods of steady frequencies from a higher frequency to a lower frequency. The higher frequency and lower frequency represent a frequency range, which spans at least a range of two frequencies corresponding to the one lower than and one higher than the resonance of the standing wave mode of the cavity or acoustic chamber. As the frequency steps through the different values as illustrated in the bottom graph in FIG. 11, the particles or cells in the acoustic standing wave are spatially shifted or translated to a new location. The new position of the particles or cells is represented by the new location of the nodes or antinodes of the acoustic standing wave after the frequency shift. The shifted frequency and attendant shift in location of the nodes or antinodes of the acoustic standing wave imposes a pressure gradient on the particles or cells to cause them to move to the new location of the nodes or antinodes of the acoustic standing wave. The waveform in the bottom graph causes the particles or cells to move to new locations represented by the frequency and the nodes or antinodes of the acoustic standing wave. These new locations can be determined for the acoustic chamber in which the acoustic standing wave is established, so that other structures or materials can be placed at those new locations to permit their interaction with the shifted particles or cells. In the bottom graph of FIG. 11, the frequency step pattern repeats, so that particles or cells can be shifted in sequence to a number of predetermined locations to permit interactions with different structures or materials in a predetermined order. It should be understood that numerous types of frequency shifting or sweeping patterns may be employed to achieve a desired positioning effect for the particles or cells in the acoustic standing wave, including, for example, ramps, steps, smooth curves, and any other pattern that achieves the desired positioning effect.

The present disclosure also discusses an apparatus or a device including a flow chamber (i.e. acoustic chamber) with an inlet and an outlet through which is flowed a mixture of a host fluid, first particles, and second particles. Two or more ultrasonic transducers are embedded in or outside of a wall of said flow chamber. When the two or more ultrasonic transducers are located outside the flow chamber wall, the thickness of the flow chamber wall can be tuned to maximize acoustic energy transfer into the fluid. The ultrasonic transducers are arranged at different distances from the inlet of the flow chamber. The ultrasonic transducers can be driven by an oscillating, periodic, or pulsed voltage signal of ultrasonic frequencies. The apparatus also includes two or more reflectors corresponding to each ultrasonic transducer located on the opposite wall of the flow chamber from to the corresponding transducer. Each ultrasonic transducer forms a standing acoustic wave at a different ultrasonic frequency. Each frequency can be optimized for a specific range of particle sizes in the fluid. Multiple types of particles could be reacted concurrently in the fluid using this type of apparatus.

The devices described herein could also be used to cause dispersion of at least one set of particle(s) within a fluid. For example, if a set of particles has settled to the bottom of a bag, the bag could be exposed to an acoustic standing wave that causes the particles to be dispersed throughout the fluid within the bag.

Acoustic streaming may also be utilized to produce particle-particle interactions. Some types of acoustic streaming are Gedeon streaming, Inner boundary-layer streaming, Eckart streaming, Jet driven streaming, boundary-layer driven streaming and Rayleigh streaming.

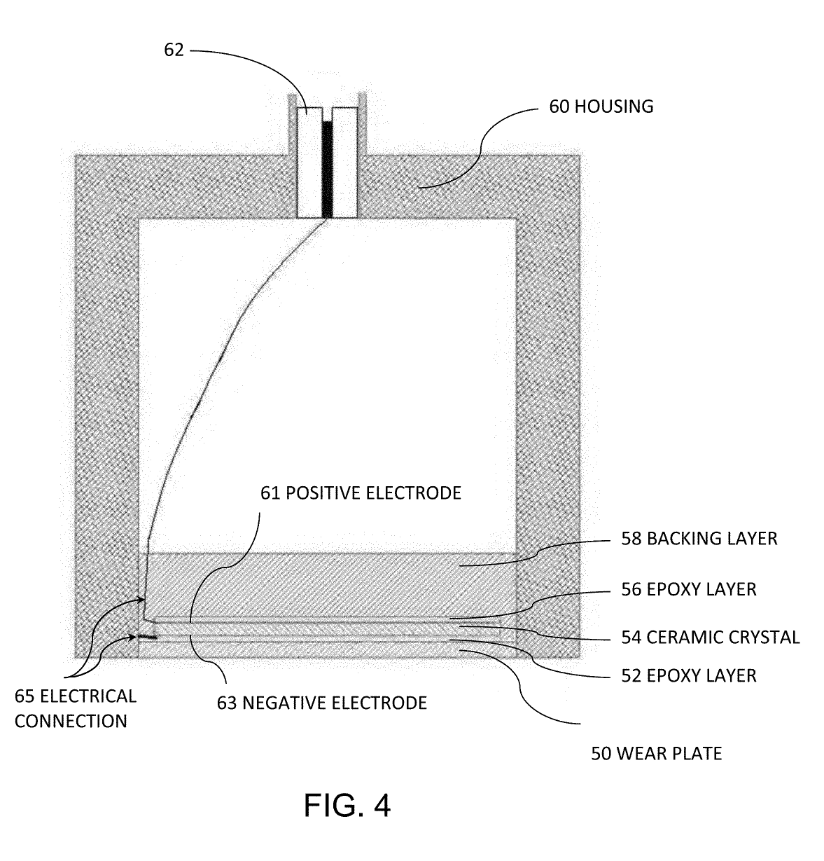

It may be helpful now to describe the ultrasonic transducer(s) used in the acoustic filtering device in more detail. FIG. 4 is a cross-sectional diagram of a conventional ultrasonic transducer. This transducer has a wear plate 50 at a bottom end, epoxy layer 52, ceramic piezoelectric element 54 (made of, e.g. Lead Zirconate Titanate (PZT) or lithium niobate), an epoxy layer 56, and a backing layer 58. On either side of the ceramic piezoelectric element, there is an electrode: a positive electrode 61 and a negative electrode 63. The epoxy layer 56 attaches backing layer 58 to the piezoelectric element 54. The entire assembly is contained in a housing 60 which may be made out of, for example, aluminum. The housing is used as the ground electrode. An electrical adapter 62 provides connection for wires to pass through the housing and connect to leads (not shown) which attach to the piezoelectric element 54. Typically, backing layers are designed to add damping and to create a broadband transducer with uniform displacement across a wide range of frequency and are designed to suppress excitation of particular vibrational eigen-modes of the piezoelectric element. Wear plates are usually designed as impedance transformers to better match the characteristic impedance of the medium into which the transducer radiates.

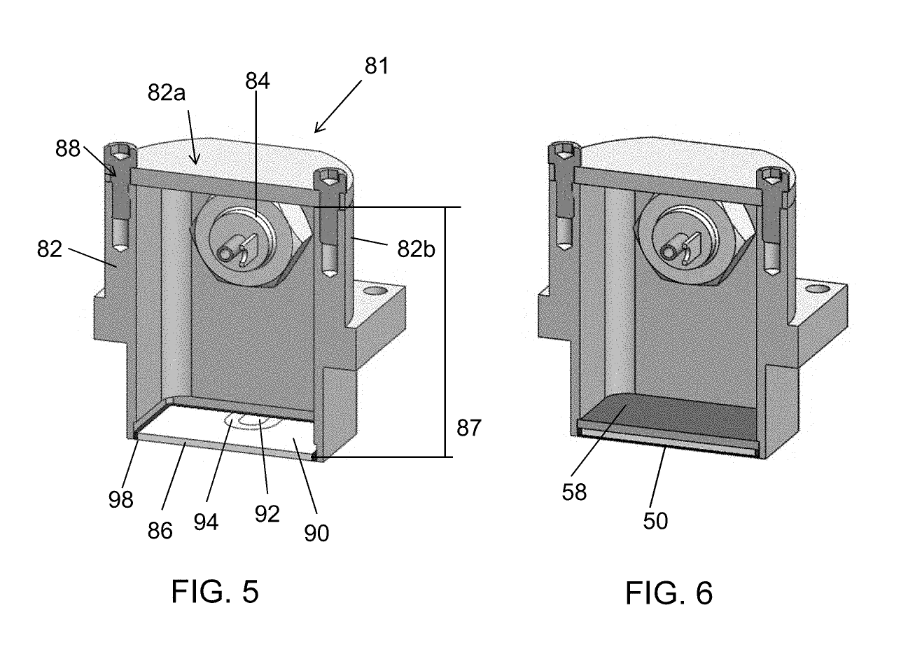

FIG. 5 is a cross-sectional view of an ultrasonic transducer 81 of the present disclosure, which is used in the acoustic filtering device of the present disclosure. Transducer 81 is shaped as a square, and has an aluminum housing 82. The aluminum housing has a top end and a bottom end. The transducer housing may also be composed of plastics, such as medical grade HDPE or other metals. The piezoelectric element is a mass of perovskite ceramic, each consisting of a small, tetravalent metal ion, usually titanium or zirconium, in a lattice of larger, divalent metal ions, usually lead or barium, and O²⁻ ions. As an example, a PZT (lead zirconate titanate) piezoelectric element 86 defines the bottom end of the transducer, and is exposed from the exterior of the bottom end of the housing. The piezoelectric element is supported on its perimeter by a small elastic layer 98, e.g. epoxy, silicone or similar material, located between the piezoelectric element and the housing. Put another way, no wear plate or backing material is present. However, in some embodiments, there is a layer of plastic or other material separating the piezoelectric element from the fluid in which the acoustic standing wave is being generated. The piezoelectric material/element/crystal has an exterior surface (which is exposed) and an interior surface as well.

Screws 88 attach an aluminum top plate 82a of the housing to the body 82b of the housing via threads. The top plate includes a connector 84 for powering the transducer. The top surface of the piezoelectric element 86 is connected to a positive electrode 90 and a negative electrode 92, which are separated by an insulating material 94. The electrodes can be made from any conductive material, such as silver or nickel. Electrical power is provided to the piezoelectric element 86 through the electrodes on the piezoelectric element. Note that the piezoelectric element 86 has no backing layer or epoxy layer. Put another way, there is an interior volume or an air gap 87 in the transducer between aluminum top plate 82a and the piezoelectric element 86 (i.e. the air gap is completely empty). A minimal backing 58 and/or wear plate 50 may be provided in some embodiments, as seen in FIG. 6.

The transducer design can affect performance of the system. A typical transducer is a layered structure with the ceramic piezoelectric element bonded to a backing layer and a wear plate. Because the transducer is loaded with the high mechanical impedance presented by the standing wave, the traditional design guidelines for wear plates, e.g., half wavelength thickness for standing wave applications or quarter wavelength thickness for radiation applications, and manufacturing methods may not be appropriate. Rather, in one embodiment of the present disclosure the transducers, there is no wear plate or backing, allowing the piezoelectric element to vibrate in one of its eigenmodes with a high Q-factor, or in a combination of several eigenmodes. The vibrating ceramic piezoelectric element/disk is directly exposed to the fluid flowing through the fluid cell.

Removing the backing (e.g. making the piezoelectric element air backed) also permits the ceramic piezoelectric element to vibrate at higher order modes of vibration with little damping (e.g. higher order modal displacement). In a transducer having a piezoelectric element with a backing, the piezoelectric element vibrates with a more uniform displacement, like a piston. Removing the backing allows the piezoelectric element to vibrate in a non-uniform displacement mode. The higher order the mode shape of the piezoelectric element, the more nodal lines the piezoelectric element has. The higher order modal displacement of the piezoelectric element creates more trapping lines, although the correlation of trapping line to node is not necessarily one to one, and driving the piezoelectric element at a higher frequency will not necessarily produce more trapping lines.

In some embodiments of the acoustic filtering device of the present disclosure, the piezoelectric element may have a backing that minimally affects the Q-factor of the piezoelectric element (e.g. less than 5%). The backing may be made of a substantially acoustically transparent material such as balsa wood, foam, or cork which allows the piezoelectric element to vibrate in a higher order mode shape and maintains a high Q-factor while still providing some mechanical support for the piezoelectric element. The backing layer may be a solid, or may be a lattice having holes through the layer, such that the lattice follows the nodes of the vibrating piezoelectric element in a particular higher order vibration mode, providing support at node locations while allowing the rest of the piezoelectric element to vibrate freely. The goal of the lattice work or acoustically transparent material is to provide support without lowering the Q-factor of the piezoelectric element or interfering with the excitation of a particular mode shape.

Placing the piezoelectric element in direct contact with the fluid also contributes to the high Q-factor by avoiding the dampening and energy absorption effects of the epoxy layer and the wear plate. Other embodiments of the transducer(s) may have wear plates or a wear surface to prevent the PZT, which contains lead, contacting the host fluid. This may be desirable in, for example, biological applications such as separating blood, biopharmaceutical perfusion, or fed-batch filtration of mammalian cells. Such applications might use a wear layer such as chrome, electrolytic nickel, or electroless nickel. Chemical vapor deposition could also be used to apply a layer of poly(p-xylylene) (e.g. Parylene) or other polymer. Organic and biocompatible coatings such as silicone or polyurethane are also usable as a wear surface. Thin films, such as a polyetheretherketone (PEEK) film, can also be used as a cover of the transducer surface exposed to the fluid with the advantage of being a biocompatible material. In one embodiment, the PEEK film is adhered to the face of the piezoelectric material using pressure sensitive adhesive (PSA). Other films can be used as well.

In some embodiments, for applications such as oil/water emulsion splitting and others such as perfusion, the ultrasonic transducer has a nominal 2 MHz resonance frequency. Each transducer can consume about 28 W of power for droplet trapping at a flow rate of 3 GPM (gallons per minute). This translates to an energy cost of 0.25 kW hr/m³. This is an indication of the very low cost of energy of this technology. Desirably, each transducer is powered and controlled by its own amplifier. In other embodiments, the ultrasonic transducer uses a square piezoelectric element, for example with 1″×1″ dimensions. Alternatively, the ultrasonic transducer can use a rectangular piezoelectric element, for example with 1″×2.5″ dimensions. Power dissipation per transducer was 10 W per 1″×1″ transducer cross-sectional area and per inch of acoustic standing wave span in order to get sufficient acoustic trapping forces. For a 4″ span of an intermediate scale system, each 1″×1″ square transducer consumes 40 W. The larger 1″×2.5″ rectangular transducer uses 100 W in an intermediate scale system. The array of three 1″×1″ square transducers would consume a total of 120 W and the array of two 1″×2.5″ transducers would consume about 200 W. Arrays of closely spaced transducers represent alternate potential embodiments of the technology. Transducer size, shape, number, and location can be varied as desired to generate desired multi-dimensional acoustic standing wave patterns.

The size, shape, and thickness of the transducer determine the transducer displacement at different frequencies of excitation, which in turn affects separation efficiency. Typically, the transducer is operated at frequencies near the thickness resonance frequency (half wavelength). Gradients in transducer displacement typically result in more trapping locations for the cells/biomolecules. Higher order modal displacements generate three-dimensional acoustic standing waves with strong gradients in the acoustic field in all directions, thereby creating equally strong acoustic radiation forces in all directions, leading to multiple trapping lines, where the number of trapping lines correlate with the particular mode shape of the transducer.

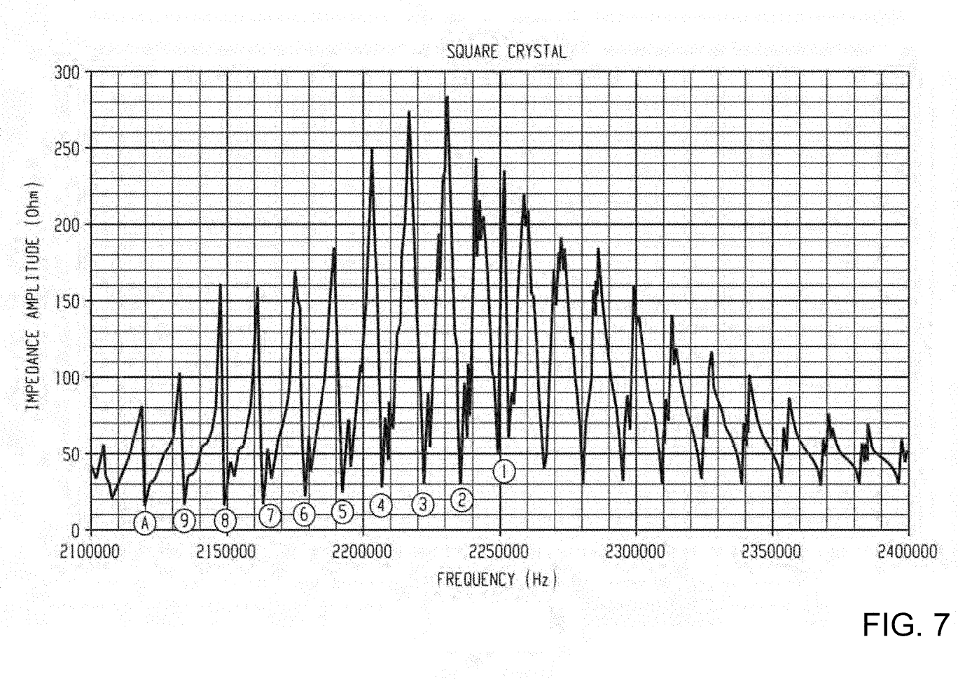

To investigate the effect of the transducer displacement profile on acoustic trapping force and separation efficiencies, an experiment was repeated ten times using a 1″×1″ square transducer, with all conditions identical except for the excitation frequency. Ten consecutive acoustic resonance frequencies, indicated by circled numbers 1-9 and letter A on FIG. 7, were used as excitation frequencies. The conditions were experiment duration of 30 min, a 1000 ppm oil concentration of approximately 5-micron SAE-30 oil droplets, a flow rate of 500 ml/min, and an applied power of 20 W. Oil droplets were used because oil is less dense than water, and can be separated from water using acoustophoresis.

FIG. 7 shows the measured electrical impedance amplitude of a square transducer as a function of frequency in the vicinity of the 2.2 MHz transducer resonance. The minima in the transducer electrical impedance correspond to acoustic resonances of the water column and represent potential frequencies for operation. Additional resonances exist at other frequencies where multi-dimensional standing waves are excited. Numerical modeling has indicated that the transducer displacement profile varies significantly at these acoustic resonance frequencies, and thereby directly affects the acoustic standing wave and resulting trapping force. Since the transducer operates near its thickness resonance, the displacements of the electrode surfaces are essentially out of phase. The typical displacement of the transducer electrodes is not uniform and varies depending on frequency of excitation. As an example, at one frequency of excitation with a single line of trapped oil droplets, the displacement has a single maximum in the middle of the electrode and minima near the transducer edges. At another excitation frequency, the transducer profile has multiple maxima leading to multiple trapped lines of oil droplets. Higher order transducer displacement patterns result in higher trapping forces and multiple stable trapping lines for the captured oil droplets.

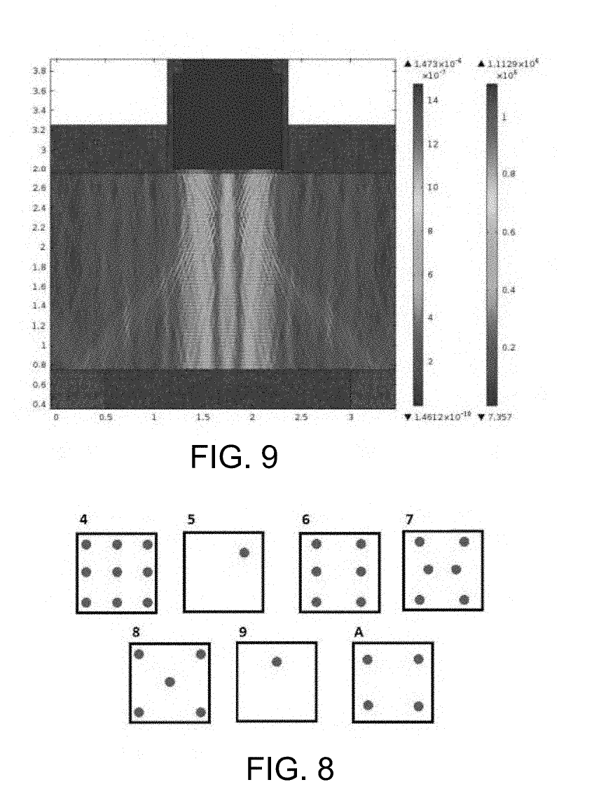

As the oil-water emulsion passed by the transducer, the trapping lines of oil droplets were observed and characterized. The characterization involved the observation and pattern of the number of trapping lines across the fluid channel, as shown in FIG. 8, for seven of the ten resonance frequencies identified in FIG. 7. Different displacement profiles of the transducer can produce different (more) trapping lines in the standing waves, with more gradients in displacement profile generally creating higher trapping forces and more trapping lines.

FIG. 9 is a numerical model showing a pressure field that matches the 9 trapping line pattern. The numerical model is a two-dimensional model; and therefore only three trapping lines are observed. Two more sets of three trapping lines exist in the third dimension perpendicular to the plane of the page.

The lateral force of the acoustic radiation force generated by the transducer can be increased by driving the transducer in higher order mode shapes, as opposed to a form of vibration where the crystal effectively moves as a piston having a uniform displacement. The acoustic pressure is proportional to the driving voltage of the transducer. The electrical power is proportional to the square of the voltage. The transducer is typically a thin piezoelectric plate, with electric field in the z-axis and primary displacement in the z-axis. The transducer is typically coupled on one side by air (i.e., the air gap within the transducer) and on the other side by the fluid mixture containing the particles that will be interacted with each other. The types of waves generated in the plate are known as composite waves. A subset of composite waves in the piezoelectric plate is similar to leaky symmetric (also referred to as compressional or extensional) Lamb waves. The piezoelectric nature of the plate typically results in the excitation of symmetric Lamb waves. The waves are leaky because they radiate into the water layer, which result in the generation of the acoustic standing waves in the water layer. Lamb waves exist in thin plates of infinite extent with stress free conditions on its surfaces. Because the transducers of this embodiment are finite in nature, the actual modal displacements are more complicated.

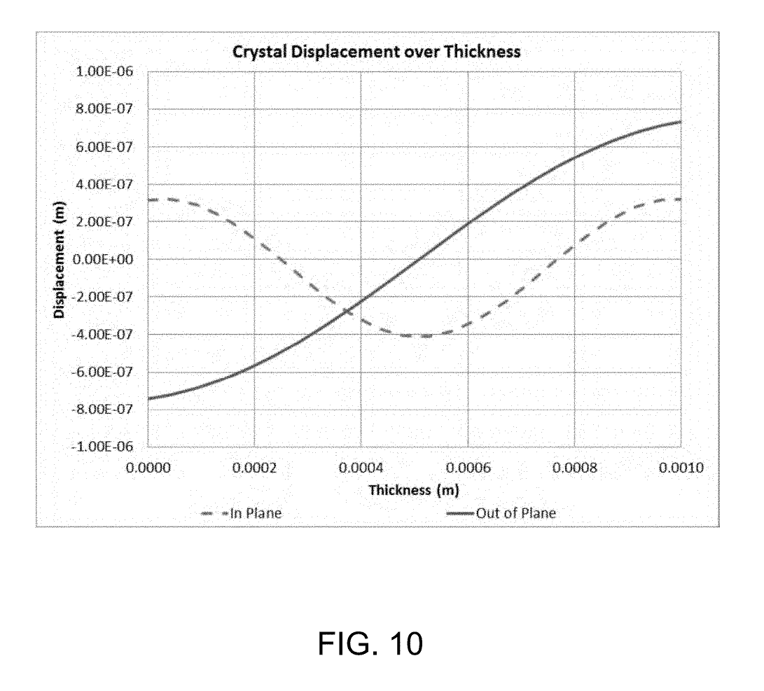

FIG. 10 shows the typical variation of the in-plane displacement (x-displacement) and out-of-plane displacement (y-displacement) across the thickness of the plate, the in-plane displacement being an even function across the thickness of the plate and the out-of-plane displacement being an odd function. Because of the finite size of the plate, the displacement components vary across the width and length of the plate. In general, a (m,n) mode is a displacement mode of the transducer in which there are m undulations in transducer displacement in the width direction and n undulations in the length direction, and with the thickness variation as described in FIG. 10. The maximum number of m and n is a function of the dimension of the piezoelectric material (e.g., a piezoelectric crystal) and the frequency of excitation. Additional three-dimensional modes exist that are not of the form (m,n).

The transducers are driven so that the piezoelectric element vibrates in higher order modes of the general formula (m, n), where m and n are independently 1 or greater. Generally, the transducers will vibrate in higher order modes than (2,2). Higher order modes will produce more nodes and antinodes, result in three-dimensional standing waves in the water layer, characterized by strong gradients in the acoustic field in all directions, not only in the direction of the standing waves, but also in the lateral directions. As a consequence, the acoustic gradients result in stronger trapping forces in the lateral direction.

Generally, the ultrasonic transducer(s) may be driven by an electrical signal, which may be controlled based on voltage, current, phase angle, power, frequency or any other electrical signal characteristic. In particular, the driving signal for the transducer may be based on voltage, current, magnetism, electromagnetism, capacitive or any other type of signal to which the transducer is responsive. In embodiments, the voltage signal driving the transducer can have a sinusoidal, square, sawtooth, pulsed, or triangle waveform; and have a frequency of 500 kHz to 10 MHz. The voltage signal can be driven with pulse width modulation, which produces any desired waveform. The voltage signal can also have amplitude or frequency modulation start/stop capability to eliminate streaming. In particular embodiments, the voltage signal can have a frequency of about 3 MHz to about 30 MHz, so that such frequencies are produced by the ultrasonic transducer.

The transducers are used to create a pressure field that generates acoustic radiation forces of the same order of magnitude both orthogonal to the standing wave direction and in the standing wave direction. When the forces are roughly the same order of magnitude, particles of size 0.1 microns to 300 microns will be moved more effectively towards “trapping lines”, so that the first particles and second particles are co-located next to each other, permitting them to react with each other.

In biological applications, all of the parts of the system (i.e., the bioreactor, acoustic filtering device, tubing fluidly connecting the same, etc.) can be separated from each other and be disposable. Avoiding centrifuges and filters allows better separation of the biological cells from fluid without lowering the viability of the cells. The transducers may also be driven to create rapid pressure changes to prevent or clear blockages due to agglomeration of biological cells. The frequency of the transducers may also be varied to obtain optimal effectiveness for a given power.

The techniques and implementations described herein may be used for integrated continuous automated bioprocessing. Control can be distributed to some or all units involved in the bioprocessing. Feedback from units can be provided to permit overview of the bioprocess, which may be in the form of screen displays, control feedbacks, reporting, status reports and other information conveyance. Distributed processing permits a high degree of flexibility in achieving a desired process control, for example by coordinating steps among units and providing a batch executive control.

The acoustophoretic devices utilizing an acoustic wave system can be implemented with biocompatible materials, and may include gamma sterilizable and single use components. The processing system also permits ultrasonic flow measurement, which is noninvasive, and is capable of operating with high viscosity fluids. The system can be implemented with single use sterile septic connectors and a simple graphical user interface (GUI) for control. The acoustophoretic device is scalable. For example, a relatively small unit is capable of operation at 2 L to 50 L scale.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

The following examples is provided to illustrate the devices and processes of the present disclosure. The examples are merely illustrative and are not intended to limit the disclosure to the materials, conditions, or process parameters set forth therein.

### EXAMPLES

### Example 1

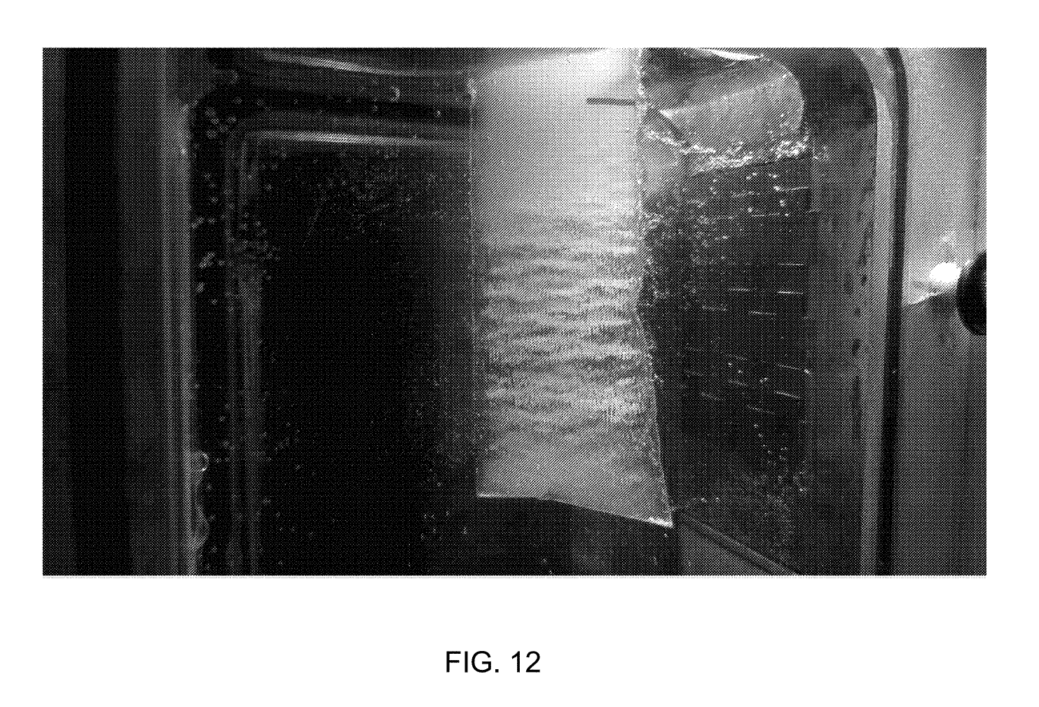

FIG. 12 is a picture of a plastic bag containing a fluid mixture with T-cells and viruses. The plastic bag was placed into an acoustophoretic device that was filled with water. A multi-dimensional acoustic standing wave was generated, causing the T-cells and viruses to interact with each other. This is visible as a series of beams of disks within the plastic bag.

### Example 2

The BacMam® system (ThermoFisher Scientific) uses baculoviruses for transduction, and was used for transduction of green fluorescent protein (GFP) into Jurkat T-cells. This system was used for various experiments. Five results are shown below. They were labeled Control, Process Control 1, Process Control 2, Acoustics 3 MHz, and Acoustics 10 MHz.

The Control experiment, the Process Control 1 experiment, and the Process Control 2 experiment were not exposed to acoustic standing waves.

For the Acoustics 3 MHz experiment, interaction between the T-cells and viruses was enhanced using an acoustic standing wave of nominal frequency 3 Hz.

For the Acoustics 10 MHz experiment, interaction between the T-cells and viruses was enhanced using an acoustic standing wave of nominal frequency 10 Hz.

The results are listed in the table below. The MOI is the multiplicity of infection, or the number of viral vector particles per cell. The GFP+ is the % of cells that expressed GFP.

| Experiment | MOI | GFP+ (%) |
|---|---|---|
| Control | — | — |
| Process Control 1 | 50 | 28.4 |
| Process Control 2 | 50 | 48.8 |
| Acoustics 3 MHz | 10 | 21.8 |
| Acoustics 10 MHz | 10 | 48.4 |

Using acoustics resulted in equivalent transduction efficiency with 80% fewer viral particles per cell.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

1. A method for causing interaction between first particles and second particles, comprising:
placing the first particles and the second particles in an acoustophoretic device comprising:
an acoustic chamber in which the first particles and the second particles are placed; and
an ultrasonic transducer including a piezoelectric material that can be driven to create a multi-dimensional acoustic standing wave in the acoustic chamber; and
driving the ultrasonic transducer to create the multi-dimensional acoustic standing wave;
wherein the first particles and the second particles are co-located by the multi-dimensional acoustic standing wave.

2. The method of claim 1, wherein the first particles and the second particles are suspended in a fluid.

3. The method of claim 1, wherein the first particles are cells, and the second particles are selected from the group consisting of antibodies, beads, and viruses.

4. The method of claim 3, wherein the cells are Chinese hamster ovary (CHO) cells, NSO hybridoma cells, baby hamster kidney (BHK) cells, human cells, regulatory T-cells, helper T-cells, cytotoxic T-cells, memory T-cells, effector T-cells, gamma delta T-cells, Jurkat T-cells, CAR-T cells, B cells, or NK cells, peripheral blood mononuclear cells (PBMCs), algae, plant cells, bacteria, or viruses.

5. The method of claim 1, wherein the ultrasonic transducer is driven for a time period of about 5 minutes to about 15 minutes.

6. The method of claim 1, wherein the ultrasonic transducer is driven at a frequency of about 3 MHz to about 20 MHz.

7. The method of claim 1, wherein the frequency of the multi-dimensional acoustic standing wave is varied in a sweep pattern to move the first particles relative to the second particles.

8. The method of claim 1, wherein the piezoelectric material of the ultrasonic transducer is lead zirconate titanate (PZT) or lithium niobate.

9. The method of claim 1, wherein the acoustophoretic device further comprises a cooling unit for cooling the ultrasonic transducer.

10. The method of claim 1, wherein the first particles and the second particles have acoustic contrast factors of the same sign.

11. The method of claim 1, wherein the first particles and the second particles have acoustic contrast factors with opposite signs.

12. A method for interacting first particles with second particles, comprising:
placing the first particles and the second particles in an acoustophoretic device comprising:
an acoustic chamber in which the first particles and the second particles are placed; and
an ultrasonic transducer including a piezoelectric material; and
driving the ultrasonic transducer to cause acoustic streaming;
wherein the acoustic streaming causes the first particles to interact with the second particles.

13. The method of claim 12, wherein the first particles and the second particles are suspended in a fluid.

14. The method of claim 12, wherein the first particles are cells, and the second particles are selected from the group consisting of antibodies, beads, and viruses.

15. The method of claim 14, wherein the cells are Chinese hamster ovary (CHO) cells, NSO hybridoma cells, baby hamster kidney (BHK) cells, human cells, regulatory T-cells, helper T-cells, cytotoxic T-cells, memory T-cells, effector T-cells, gamma delta T-cells, Jurkat T-cells, CAR-T cells, B cells, or NK cells, peripheral blood mononuclear cells (PBMCs), algae, plant cells, bacteria, or viruses.

16. The method of claim 12, wherein the ultrasonic transducer is driven for a time period of about 5 minutes to about 15 minutes.

17. The method of claim 12, wherein the ultrasonic transducer is driven at a frequency of about 3 MHz to about 20 MHz.

18. The method of claim 12, wherein the piezoelectric material of the ultrasonic transducer is lead zirconate titanate (PZT) or lithium niobate.

19. The method of claim 12, wherein the acoustophoretic device further comprises a cooling unit for cooling the ultrasonic transducer.

20. A method for dispersing at least one set of particles throughout a host fluid, comprising:
placing the at least one set of particles and the host fluid in an acoustophoretic device comprising:
an acoustic chamber in which the at least one set of particles and the host fluid are placed; and
an ultrasonic transducer including a piezoelectric material; and
driving the ultrasonic transducer to cause the at least one set of particles to be dispersed throughout the host fluid.
